(19) United States
(12) Patent Application Publication
(10) Pub. No.: US 2016/0008528 A1
(43) Pub. Date: Jan. 14, 2016

(54) LOW COST SPINNING AND FABRICATION OF HIGH EFFICIENCY (HE) HAEMODIALYSIS FIBERS AND METHOD THEREOF

(71) Applicant: Indian Institute of Technology, Kharagpur, West Bengal (IN)

(72) Inventors: Anirban Roy, West Bengal (IN); Sirshendu De, West Bengal (IN); Lloyd Vincent, West Bengal (IN); Shyam Vasudeva Rao, West Bengal (IN)

(21) Appl. No.: 14/598,697

(22) Filed: Jan. 16, 2015

(30) Foreign Application Priority Data
Jul. 11, 2014 (IN) 754/KOL/2014

(51) Int. Cl. A61M 1/16; D01D 5/38; B29C 47/00; D01D 5/06

(52) U.S. Cl. CPC A61M 1/1623; D01D 5/06; D01D 5/38; B29C 47/0023; B29C 47/0014; B29L 2031/7546

(57) ABSTRACT

The present invention relates to an apparatus and a method to spin hollow fibres of dialysis grade with diameter around 220 microns and thickness of around 35-40 microns, by wet spinning technique. The present invention spinning is carried out by using an apparatus having a nitrogen cylinder (1), water bucket (2), polymer cylinder (6), water cylinder (7), automatic winding machine (5) characterized by a cheap assembly of syringes (dispo van (8) and insulin syringes (9)) wherein no electrical power is required for the spinning, making the fibres extremely easy to manufacture and affordable at the consumer end.

### FIELD OF THE INVENTION

The present invention relates to an apparatus and method to spin hollow fibres of dialysis grade. More particularly, the present invention relates to spin hollow fibres of dialysis grade with diameter around 220 microns and thickness of around 35-40 microns, by wet spinning technique.

### BACKGROUND OF THE INVENTION

Typically, patients suffering from end stage renal disease (ESRD) are administered treatment through dialysis. Dialysis is of two types: (i) Peritoneal dialysis, and (ii) Hemodialysis. In India, hemodialysis is carried out using dialysis cartridges, which are imported from Japan, Germany and Korea, since the expertise of manufacturing these is not presently available. The crux of the problem lies in spinning of hollow fibers, of specific dimensions, and potting them in a casing. Typically, individual such hollow fibres would measure around 180-220 microns in diameter, with wall thickness of around 15-35 microns. Around 7000-15000 of such individual hollow fibres, each 24 cm in length, are potted together in a casing giving a surface area of around 1 m² for the dialysis operation. The dimensions of the hollow fibres are of utmost importance, since the priming volume should be minimum while yielding the required surface area. The methodology of producing the commercial hollow fibres involves the use of an expensive spinneret based technology. This makes them expensive, and import taxes and other related costs make the cost of such one dialysis cartridge to be in the range of 1500-2500 INR. A typical patient requires dialysis operation 3 times a week, which means using 3 such cartridges per week and 12 such cartridges per month. Due to economic constraints, majority of the people are unable to use 3 cartridges per week, resulting to two-three reuses of the same cartridge.

Dialysis fibre manufacturing is a proprietary technology of the manufacturing big houses like Nipro, Asahi and Fresenius. The patented technology is basically about producing hollow fibres of 180-220 micron inner diameter with thickness ranging from 15-35 microns. There is a lot of reported literature in case of hollow fibre spinning in general, both wet and dry spinning. However, very few such exist when it comes to dialysis grade hollow fibres since developing such a technology is very challenging and of course controlling such a technology lets the inventor enjoy the monopoly in the market. Usually the spinning of hollow fibres is achieved through the expensive spinneret based technique which in turn makes the fibres expensive. There are various ways to extrude hollow fibres, like, of course conventional spinnerets, capillary channels, custom made extrusion heads. As far as the material specification of the dialysis fibres is concerned, historically, cellulose acetate was the initial choice for such application primarily due to the fact that it was a naturally occurring polymer. However, gradually due to complications arising during dialysis procedure, like blood incompatibility, and advanced materials engineering led scientists to use polymers like polyacrylonitrile, polysulfone, polymethylmethacrylate etc.

U.S. Pat. No. 4,483,903A discloses a preparation of hollow acrylonitrile fibres and filaments by dry spinning the spinning dope through a nozzle having loop-shaped nozzle orifices where the solution having a viscosity equivalent to at least 120 falling ball seconds, measured at 80° C., or at least 75 falling ball seconds, measured at 100° C., wherein the nozzle orifice area of the profiling nozzle is smaller than 0.2 mm² and the maximum width of the sides of the loop-shaped nozzle is 0.1 mm and the overlap between the two ends of the sides of the loop-shaped nozzle forms an angle of from 10° to 30° measured from the center of the nozzle and wherein the spinning air acts on the filaments in a transverse direction to the filament take-off and the air direction forms an angle of from 80° to 100° with a straight line passing through the opening between the sides.

The journal “Preparation of Polyvinylidene Fluoride (PVDF) Hollow Fiber Hemodialysis Membranes” by Qinglei Zhang, Xiaolong Lu and Lihua Zhao, discloses that PVDF hollow fiber membranes were prepared by non-solvent-induced phase separation (NIPS) through spinning equipment which contains dope tank, metal filter, liquid tank, flow meter, spinneret, agitator, nitrogen cylinder, coagulation bath, take-up wheel, control device. The article further teaches about an apparatus for determining the maximum pore size of the hollow fiber membranes using nitrogen bottle, regulator, precise pressure gauge, valve, container, syringe needles, transparent cylinder, PVDF membrane sample to be tested in absolute ethyl alcohol.

The paper “Polysulphone and Polyethersulphone Hollow Fiber Membranes with Developed Inner Surface as Material for Bio-medical Applications” by Andrzej Chwojnowski, Cezary Wojciechowski, Konrad Dudziński, Ewa Lukowska discloses that the preparation of hollow fiber membranes by means of a home-made device with replaceable spinnerets. In the coagulation and washing baths during membrane formation and washing process constant water flow was maintained. The polymer solution was delivered from a chamber to the external spinneret nozzle by a spinning pump, whereas the core fluid (water) to the central nozzle under the pressure of nitrogen from the cylinder (666.6-4444 Pa or 5-40 mmHg). The size of the air gap was 13-29 cm from the water level. The temperature of the membrane forming mixtures, air and spinneret was 21-22° C. The temperature of water in the coagulation and washing baths was 20±1° C. Relative humidity of air in the air gap was controlled in the range from 60 up to 99%. The article further teaches that by selecting appropriate parameters of the spinning process it is possible to obtain semi-permeable hollow fibers of developed inner surface by means of standard spinnerets. The key parameters are core liquid pressure, membrane-forming mixture pressure, air gap size, relative humidity in air gap and stable temperature. By appropriate selection of these parameters the formation of the membrane inner corrugation can be controlled. There is a possibility of obtaining membranes with very dense and fine corrugation as well as membranes with several larger corrugations.

U.S. patent no. 20120213998A1 discloses about a process for the preparation of a hollow filament (F) based on one or several molten or dissolved hyper-branched polymers (P) and potentially one or several further polymers (FP), characterized in that the molten or dissolved hyper-branched polymer (P) or the mixture of the hyper-branched polymer (P) with the further polymer (FP) is passed through one or several spinnerets (S), wherein the ratio between the spinneret die-length (L) and the die-channel (Delta-D) is between 0.1 and 9.5. The process can be applied for the preparation of hyper-branched polyethersulfone (HPES) hollow filaments. The document also teaches that the dope solution and bore-fluid were extruded at a specified flow rate through a spinneret using two ISCO syringe pumps.

The prevailing technology for spinning dialysis fibres involves the use of expensive spinnerets. These spinnerets cost thousands of dollars. However, just buying the spinnerets of required dimensions do not guarantee the ability to spin dialysis grade hollow fibres, else the ability to spin such fibres would not be restricted to just a handful of companies around the world. It is the patented technology, and modification(s) of these spinnerets, of these manufacturing big houses which makes the technology so clandestine.

Although the arrangements of the single syringe or multiple syringes were known in the prior art to build spinnerets but there is a need of a cheap arrangement to spin fibres of dialysis grade so that the hollow fibres can be manufactured easily and affordable at the consumer end.

The present invention provides a method of spinning dialysis grade hollow fibres using simple low cost needle assembly as well as a power free process.

### OBJECT OF THE INVENTION

The primary object of the present invention is to provide a design of apparatus to spin hollow fibres of dialysis grade.

Another object of the present invention is to provide a method of spinning dialysis grade hollow fibres using simple needle assembly.

Another object of the present invention is to provide hollow fibres of dialysis grade with diameter around 220 microns and thickness of around 35-40 microns, by wet spinning technique.

Another object of the present invention is to provide spinning of hollow fibres by cheap assembly of syringes using dispo van and insulin syringe.

Yet another object of the present invention is to provide a method of spinning dialysis grade hollow fibres where no electrical power is required for spinning.

Another object of the present invention to enable preparation of dialysis range hollow fibres using different polymers/polymeric blends/surface modified fibres.

Another object of the present invention is to provide a method of making the fibres extremely easy to manufacture and affordable at the consumer end.

### SUMMARY OF THE INVENTION

Dialysis hollow fibres have internal diameter 180-220 microns, with wall thickness 30-35 microns. Dimensions of fibres are important, since the priming volume should be minimum with required surface area. Production of commercial fibres involves spinneret based technology. This makes them expensive and final cost of the cartridge is in the range of Rs. 1500-2500. A typical patient requires dialysis 3 times a week. Due to economic constraints, majority of the people are unable to use 3 cartridges per week, resulting to two-three reuses of the same cartridge. The present invention discusses low cost needle based spinning technology for such fibers.

The present invention serves a twofold purpose. First is the proposing a new design to spin hollow fibres of dialysis grade with diameter around 220 microns and thickness of around 35-40, microns, by wet spinning technique. The second novel aspect is the fact that spinning is carried out by cheap assembly of syringes (dispo van and insulin syringes) and moreover, no electrical power is required for the spinning, making the fibres extremely easy to manufacture and affordable at the consumer end.

The present invention discloses an apparatus to spin hollow fibres of dialysis grade by wet spinning technique wherein no electrical power is required for the spinning comprising cylinder containing gas, bucket means, automatic winding machine, an assembly of needles characterized by the assembly of needles which are dispo van and insulin syringes wherein the outer needle is a dispo van syringe with the inner needle being insulin syringe.

The outer needle is a dispo van size 22 needle (around 700 microns diameter) and the inner needle being a 32 gauge insulin needle (BD ultra fine pen needle, around diameter of 230 microns) wherein the inner needle is bent at an angle of 120° and inserted into the outer needle. The inner needle is used to flow anti solvent used for phase inversion which is water in the present invention. The outer needle and inner needle forms a shell and the polymer flows through a ball valve, which is employed in the polymer cylinder to control polymer flow rate with an on/off type arrangement, to the shell. Whereas the water flows through a water cylinder via a needle valve, employed in water cylinder for the control of water flow rate minutely, to the inner needle. The pressure is maintained at about 20-30 psi (140 to 200 kPa) during the entire spinning duration with the help of a cylinder containing gas. The gelation bath used for the spinning process is normal tap water and the wet spun fibres are then wound on the spool using the automatic winding machine.

The present invention enables preparation of dialysis range hollow fibres using different polymers/polymeric blends/surface modified fibres. Scaling up to the industrial level is also not a great deal of challenge due to extreme simplicity of design proposed.

### BRIEF DESCRIPTION OF ACCOMPANYING FIGURES

Various structures are schematically depicted in the drawings for purposes of explanation only and so as to not obscure the present invention with details that are well known to those skilled in the art. Nevertheless, the attached drawings are included to describe and explain illustrative examples. The drawings are not to scale and are intended for use in conjunction with the explanations in the following detailed description.

FIG. 1 illustrates the schematic representation of a hollow fiber spinning unit.

FIG. 2 illustrates the polymer and water cylinder assemblies.

FIG. 3 illustrates the needle assembly.

FIGS. 4A, 4B, 4C and 4D illustrates the scanning electron microscope images of the dialysis grade fibres obtained from the spinning.

FIG. 5A illustrates the hydraulic permeability of the membrane.

FIG. 5B illustrates the Molecular Weight Cut Off of various dialysis fibres

FIG. 6 illustrates the Urea and Creatinine clearances with feed flow rate.

FIG. 7 illustrates the Kt/V values of the dialysis membrane.

FIG. 8A illustrates the urea filtration for various feed and dialysate flow rates.

FIG. 8B illustrates creatinine filtration for various feed and dialysate flow rates.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure.

Throughout the drawings, it should be noted that like reference numbers are used to depict the same or similar elements, features, and structures.

### DETAILED DESCRIPTION OF THE INVENTION

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

The present invention provides an apparatus and a method of spinning dialysis grade hollow fibres using simple needle assembly wherein no electrical power is required for the spinning producing hollow fibres of diameter around 220 microns and thickness of around 35-40 microns.

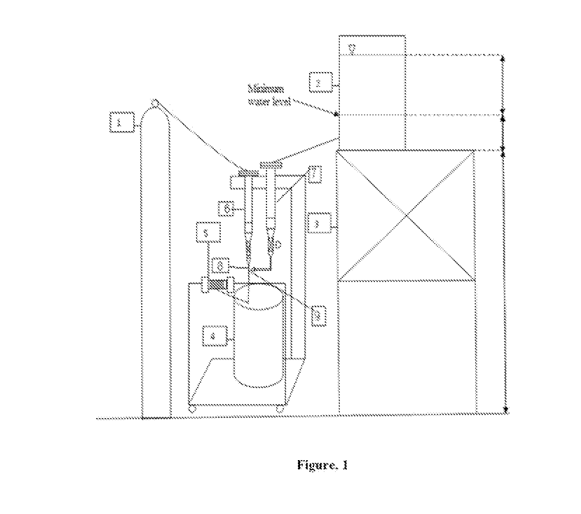

FIG. 1 is a schematic representation of a hollow fiber spinning unit in which the cylinder containing gas (1) is used to pressurize the polymer through the syringe assembly, where the gas may be inert gas like nitrogen. The pressure is maintained at about 20-30 psi (140 to 200 kPa) during the entire spinning duration. The anti solvent used for phase inversion is water. Water is stored in a bucket means (2) and is kept at a height of 120 cm (4 feet from the ground). The minimum water level to be maintained for the spinning of the fibres is 145-155 cm from the ground level. Generally, the level of water is maintained at 150-160 cms during the spinning process. The water flows due to the gravitational head through the cylinders (polymer cylinder (6) and water cylinder (7)) and the needle assembly (dispo van syringe (8) and insulin syringe (9)). The water flow rate at the outlet of the syringe is about 0.2 ml/min. The gelation bath (4) used for the spinning process is normal tap water and the wet spun fibres are then wound on the spool using the automatic winding machine (5).

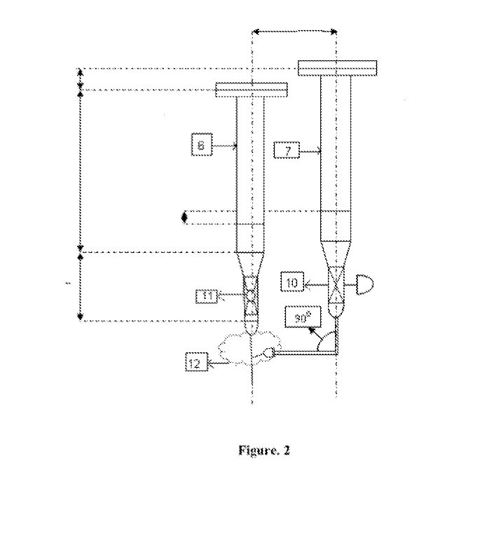

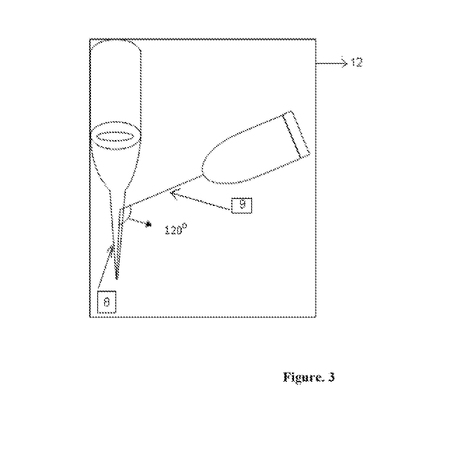

FIG. 2 describes the polymer cylinder (6) and water cylinder (7) assemblies. The flanges of both the cylinders are 9.5 cm in diameter. The water cylinder (7) is 9.5 cm longer than the polymer cylinder (6), with the flange to flange distance between the two being 9.5 cm. The polymer flow rate basically requires an on/off type arrangement, hence a ball valve (11) is employed. However, since there might be a necessity for the water flow rate to be minutely controlled, hence a needle valve (10) is employed for its control. The water flows from the water cylinder (7) to the needle assembly (12) through a pipe bend at 90° angle. The needle assembly (12) is discussed in the FIG. 3. The centre to centre distance of the cylinders is ensured to 12.5 cm throughout the assembly.

FIG. 3 describes the heart of the spinning, i.e. the needle assembly. The outer needle (8) is a dispo van size 22 needle (700 microns diameter) with the inner one (9) being a 32 gauge insulin needle (BD ultra fine pen needle, diameter of 230 microns). The inner needle (9) is bent at an angle of 120° and inserted into the outer needle (8). The needles are sealed with m-seal to prevent any leakage and withstand the tensions/stresses developed during spinning. In this context it is to be noted that the present invention uses m-seal as sealing material however, other sealing materials can be used. Such sealing materials are not discussed herein as a person skilled in the art would be aware of the sealing material which can be used. Use of any other sealing materials will still be considered to be falling within the scope of the present invention. The water flows through the inner needle (9) and the polymer flows through the shell between the outer (8) and inner needle (9).

In the present invention describes the method of spinning of hollow fibres of dialysis grade by wet spinning technique wherein the polymer is pressurized through the syringe assembly using cylinder containing gas (1), where the gas used may be any inert gas like nitrogen. The water is stored water in a bucket means (2) as anti-solvent for phase inversion and flowing of water take place due to the gravitational head through the water cylinder (7) and the needle assembly (12) through the inner needle (9) whereas the flowing of polymer takes place through the polymer cylinder (6) and the shell formed by outer needle (8) and inner needle (9). Then gelation bath (4) is applied for the spinning process using normal tap water and wet spun fibres are wound on the spool using the automatic winding machine (5).

The polymer composition used to get the dialysis grade fibres were polysulfone(Psf):polyvinylpyrrolidone(PVP):poly ethyleneglycol(PEG) in the weight ratio of 18:1:3, dissolved in dimethyl formamide (DMF) where the molecular weight of PVP used is 40,000.

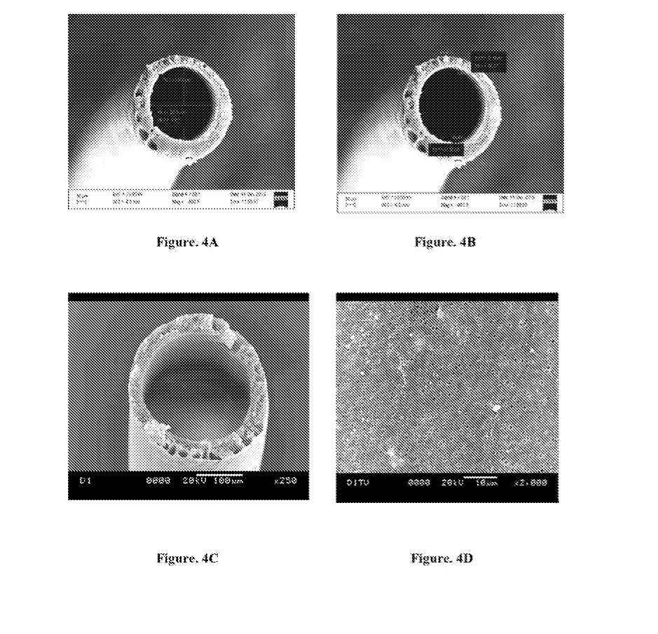

FIG. 4 illustrates the scanning electron microscope images of the dialysis grade fibres obtained from the spinning. FIG. 4A shows the cross section with the dimensions and FIG. 4B shows the thickness of the same with the dimensions. It is clear that the spun hollow fibres have an inner diameter of 224 microns and thickness of 39 microns. FIG. 4C illustrates another cross section and FIG. 4D illustrates the top view of the lateral outside surface of the membrane.

### Example 1

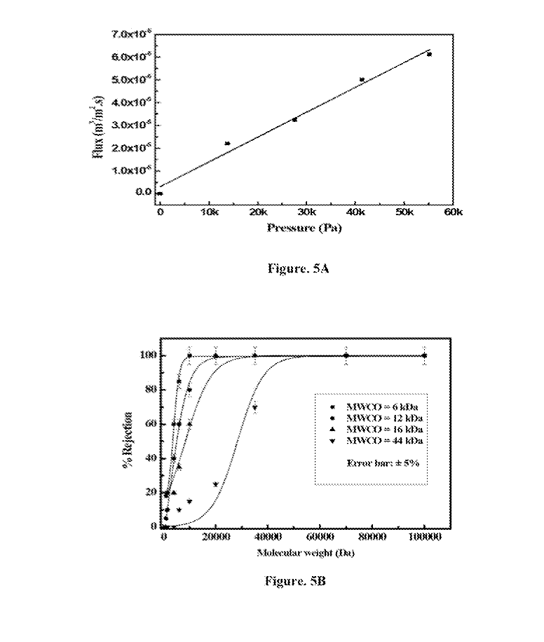

In the present example, the water permeability is measured by using pure distilled water at various transmembrane pressure drops. The permeate flux is measured at various transmembrane pressure drops and flux versus pressure drop data are plotted. This results into a straight line through origin as pure distilled water does not have any osmotic pressure. The slope of this curve gives the value of membrane permeability. It indicates how porous the hollow fiber is. This is represented in FIG. 5A.

### Example 2

In this present example, experiments are conducted using various solutes, like, polyethylene glycol of various molecular weights (400 to 35,000), dextran (70,000), etc., at 1,000 ppm and at about 12 kPa pressure and 20 l/h cross flow rate. The rejection values were measured by using the following formula:

$$R = ( 1 - \frac{{c}_{p}}{{c}_{0}} ) \times 100 \% \qquad ( 1 )$$

FIG. 5B represents the data. It is observed that the 90% rejection of solutes occur at around 6000 Da (6 kDa), hence by definition, this is the MWCO of the spun membrane.

In fact, varying the wt. % of PVP, a variety of membranes of the specified dialysis grade can be spun, the details are given in Table 2.

TABLE 2
Range of Molecular Weight Cut Off (MWCO)

| Sl. No | PVP wt. % | PEG wt. % | MWCO |
|---|---|---|---|
| 1 | 1 | 3 | 6 |
| 2 | 2 | 3 | 14 |
| 3 | 3 | 3 | 17 |
| 4 | 3 | 0 | 44 |

### Example 3

In this example, the diffusive permeabilities (P_{D}) values of the membrane is calculated. It is calculated as:

$${P}_{D} = \frac{\{ ln \frac{Δ {C}_{1} ( {t}_{1} )}{Δ {C}_{2} ( {t}_{2} )} \}}{S \{ \frac{1}{{V}_{a}} + \frac{1}{{V}_{b}} \} ( {t}_{2} - {t}_{1} )} \qquad ( 2 )$$

where, ΔC(t) is the difference between the solute concentrations in the solutes and dialysate reservoirs at the sampling time, t1 and t2, V is the reservoir volume; S is the surface area of the membrane, t is the time

The experiments are conducted in usual dialysis mode and the PD values obtained are reported in the following table:

TABLE 3
Diffusive permeabilties of the membrane

| | Polymer Blend Membrane | Reported Values |
|---|---|---|
| Components | (×10⁻⁴), cm/s | (×10⁻⁴), cm/s |
| Urea | 14.1 | 15.2 |
| Creatinine | 7.4 | 8.8 |
| Sucrose | 1.9 | 2.4 |
| Vitamin B12 | 0.22 | 0.25 |
| BSA | 0 | 0 |

### Example 4

In this example, the urea and creatinine clearances (C_{L}) are calculated as:

C_{L}=((C_{Bi}−C_{B0})/C_{Bi})×Q_{B} (3)

where, C_{Bi} and C_{B0} are the inlet and outlet concentrations of the dialyzer respectively and Q_{B} is the blood flow rate (ml/min).

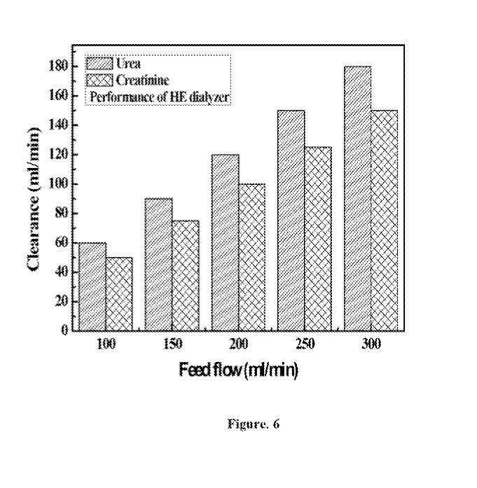

This is represented in FIG. 6. The clearances are calculated for various blood flow rates for both urea and creatinine.

### Example 5

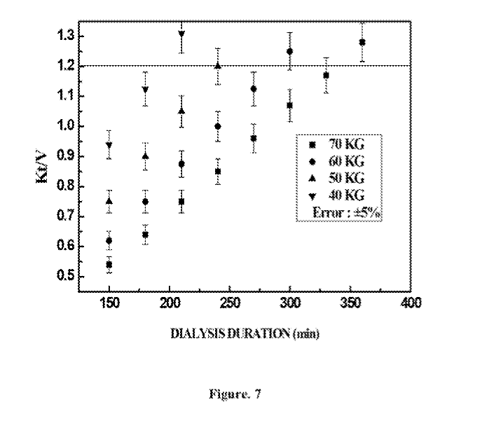

In this example, the Kt/V of the hollow fibres is found out. In this regard, K is the dialyzer clearance, expressed in milliliters per minute (mL/min) t stands for time and V is the volume of water a patient's body contains. For a good dialysis procedure, the Kt/V values should be around 1.2. Thus FIG. 7 represents these values and the performance of the fibres is gauged against various body weights.

### Example 6

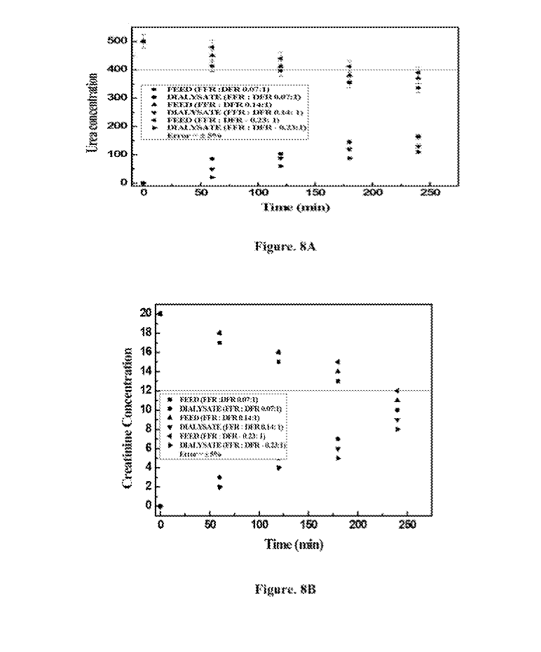

In this example, the performance of the fibres is found out, where the experiments are carried out with urea and creatinine dissolved in distilled water. The effect of the feed and dialysate flow rates is examined. The results are represented in FIG. 8A and FIG. 8B.

### Example 7

In this example, the K_{UF} values of the membranes are calculated. K_{UF} is defined as the pressure required to generate given volume of ultrafiltrate per unit time. This is found to be 10 mL/h/mmHg.

It is to be understood that the description is intended to cover all of the generic and specific features of the embodiments described herein and all the statements of the scope of the embodiments which as a matter of language might be said to fall there between.

We claim:

1. An apparatus to spin hollow fibres of dialysis grade, said apparatus comprising a cylinder containing gas, a bucket means for storage and/or collection of water,
an automatic winding machine, an assembly of needles characterized in that the said assembly of needles comprising dispo van syringe having a dispo van needle defining an outer needle; and
insulin syringe having an insulin needle defining an inner needle inserted inside said outer needle at an appropriate angle.

2. An apparatus as claimed in claim 1, wherein the said inner needle is bent at an angle of about 120° and inserted into the said outer needle.

3. An apparatus as claimed in claim 1, wherein the said outer needle is a dispo van size 22 needle having around 700 microns diameter.

4. An apparatus as claimed in claim 1, wherein the said inner needle is a 32 gauge insulin needle having around diameter of 230 microns.

5. An apparatus as claimed in claim 1, wherein the said inner needle is adapted to carry anti solvent used for phase inversion.

6. An apparatus as claimed in claim 1, wherein the said outer needle and said inner needle arrangement defines a shell through which the polymer flows.

7. An apparatus as claimed in claim 1, wherein the said needles are sealed with so as to prevent any leakage and withstand the tensions/stresses developed during spinning.

8. An apparatus as claimed in claim 1, wherein the said cylinder containing gas comprises inert gas like nitrogen.

9. An apparatus as claimed in claim 1 further comprising a polymer cylinder and water cylinder assemblies.

10. An apparatus as claimed in claim 1 and claim 9 comprising a ball valve employed in said polymer cylinder to control polymer flow rate with an on/off type arrangement.

11. An apparatus as claimed in claim 1 and claim 9 further comprising a needle valve employed in said water cylinder for the control of water flow rate minutely.

12. An apparatus as claimed in claim 1 and claim 9, wherein said polymer cylinder and said water cylinder assembly comprising flanges of about 9.5 cm in diameter.

13. An apparatus as claimed in claim 1 and claim 9, wherein the said water cylinder having greater length than the said polymer cylinder.

14. An apparatus as claimed in claim 1 and claim 9 further comprising a pipe bent at about 90° for water flow from the said water cylinder to the said assembly of needle.

15. An apparatus as claimed in claim 1 and claim 9, wherein the centre to centre distance of the said cylinders is ensured to about 12.5 cm throughout the assembly.

16. An apparatus as claimed in claim 1, wherein the said apparatus producing hollow fibres of diameter around 220 microns and thickness of around 35-40 cm.

17. A method of spinning hollow fibres of dialysis grade comprising the steps of:
a) pressurizing the polymer through the syringe assembly using nitrogen cylinder;
b) storing water as anti-solvent for phase inversion in a water bucket at a height of around 120 cm;
c) maintaining a minimum water level of around 145-155 cm from the ground level for the spinning of the fibres;
d) flowing of water due to the gravitational head through the water cylinder and the needle assembly;
e) flowing of said polymer through the polymer cylinder and the shell formed by outer needle and inner needle
f) flowing of water from the said inner needle;
g) applying gelation bath for the spinning process using normal tap water; and
h) winding wet spun fibres on the spool using the automatic winding machine.

18. The method as claimed in claim 17, wherein pressure is maintained at about 20-30 psi (140 to 200 kPa) during the entire spinning duration using said nitrogen cylinder.

19. The method as claimed in claim 17, wherein the water flow rate at the outlet of the said needle assembly is about 0.2 ml/min.

20. The method as claimed in claim 17, wherein the polymer composition used to get the dialysis grade fibres were polysulfone(Psf):polyvinylpyrrolidone(PVP):poly ethyleneglycol(PEG) in the weight ratio of 18:1:3, dissolved in dimethyl formamide (DMF).

21. The method as claimed in claim 17, wherein the molecular weight of polyvinylpyrrolidone (PVP) is 40,000.

22. The method as claimed in claim 17, wherein preparation of dialysis range hollow fibres using different polymers/polymeric blends/surface modified fibres can be done.
